(19) United States
(12) Patent Application Publication
(10) Pub. No.: US 2018/0362531 A1
(43) Pub. Date: Dec. 20, 2018

(54) NOVEL KYNUREININE PATHWAY INHIBITORS

(71) Applicant: STANDARD LLC, Los Angeles, CA (US)

(72) Inventor: Junhu ZHANG, San Diego, CA (US)

(21) Appl. No.: 16/011,432

(22) Filed: Jun. 18, 2018

Related U.S. Application Data
(60) Provisional application No. 62/522,055, filed on Jun. 19, 2017.

(51) Int. Cl. C07D 487/04

(52) U.S. Cl. CPC C07D 487/04

(57) ABSTRACT

The present disclosure provides novel compounds capable of functioning as inhibitors of the kynurenine pathway and kits.

### CROSS-REFERENCE TO RELATED APPLICATION

This application claims the benefit of U.S. Provisional Application No. 62/522,055, filed Jun. 19, 2017, which is incorporated by reference herein in its entirety.

### BACKGROUND

The kynurenine pathway plays a critical role in the human immune response. Indoleamine 2,3-dioxygenase 1 (IDO1), tryptophan 2,3-dioxygenase (TDO), and indoleamine 2,3-dioxygenase 2 (IDO2), all catalyze the first step in the kynurenine pathway by metabolizing L-tryptophan, but have only a limited structural similarity. IDO1, TDO, and IDO2 have become attractive targets for anticancer therapy. The kynurenine pathway plays a central role in the ability of a tumor cell to escape an immune response. However, a significant number of known IDO1 inhibitors show promiscuous IDO1 inhibitory activity through nonspecific mechanisms and cause undesirable side reactions. Also, many of these IDO1 inhibitors possess low micro-molar activities or limited pharmacokinetics. The design of IDO inhibitors with high enzymatic activity, high bioavailability and low toxicity remains to be very difficult. The importance of stabilizing and activating tumor immune surveillance is an established aspect of anti-cancer therapy. Hence, there is a need in the field to find new potent kynurenine pathway inhibitors, such as IDO1, IDO2, or TDO.

### SUMMARY

One aspect of the present disclosure relates to compounds comprising a structure of Structure I including pharmaceutically acceptable solvates, pharmaceutically acceptable prodrugs, pharmaceutically acceptable salts and pharmaceutically acceptable stereoisomers thereof. Other aspects relate to compositions, pharmaceutical formulations, and kits comprising the compounds disclosed herein.

### DETAILED DESCRIPTION

Most of the currently known small molecule compounds that inhibit indoleamine 2,3-dioxygenase 1 (IDO1) have weak or promiscuous inhibitory activity and undesireable side effects. Hence, there is a need for more effective IDO1 inhibitors.

I. Compounds

One aspect of the present disclosure relates to compounds comprising a structure of Structure I, including pharmaceutically acceptable solvates, pharmaceutically acceptable prodrugs, pharmaceutically acceptable salts and pharmaceutically acceptable stereoisomers thereof. In certain embodiments, the compounds provided herein are IDO1 (indoleamine 2,3-dioxygenase-1) inhibitors. In certain embodiments, the compounds provided herein are TDO inhibitors. In another embodiment, the compounds provided herein are kynurenine pathway inhibitors.

Structure I

In certain embodiments, the compounds provided herein comprise a structure of Structure I:

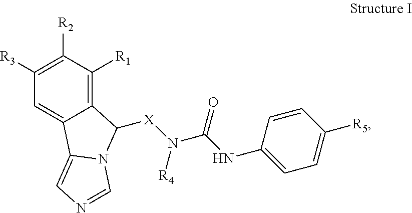

including pharmaceutically acceptable solvates, pharmaceutically acceptable prodrugs, pharmaceutically acceptable salts and pharmaceutically acceptable stereoisomers thereof, wherein:
R₁-R₃ are each independently selected from the group consisting of hydrogen, halogen (e.g. F, Cl, Br, and I), haloalkyl (e.g. CF₃), and methyl;
R₄ are each independently selected from the group consisting of hydrogen and methyl;
X is —(CH₂)ₙ—, wherein, n is 1, 2, or 3; and
R₅ is independently selected from the group consisting of halogen (e.g. F, Cl, Br, and I), haloalkyl (e.g. CF₃), methyl (—CH₃), cyano (—CN), ethynyl (—CCH), and methoxy (—OCH₃).

In certain of these embodiments, the compounds are IDO1 (indoleamine 2,3-dioxygenase-1) inhibitors.

In certain embodiments, R₁-R₄ are hydrogen, X is —(CH₂)ₙ—, wherein, n is 1, 2, or 3, and R₅ is cyano (—CN).

In certain embodiments, R₁-R₄ are hydrogen, X is —(CH₂)ₙ—, wherein, n is 1, 2, or 3, and R₅ is ethynyl (—CCH).

In certain embodiments, R₁-R₄ are hydrogen, X is —(CH₂)ₙ—, wherein, n is 1, 2, or 3, and R₅ is methyl (—CH₃).

In certain embodiments, R₁-R₄ are hydrogen, X is —(CH₂)ₙ—, wherein, n is 1, 2, or 3, and R₅ is a halogen (e.g. F, Cl, Br, and I).

In certain embodiments, R₁-R₄ are hydrogen, X is —(CH₂)ₙ—, wherein, n is 1, 2, or 3, and R₅ is haloalkyl (e.g. CF₃).

In certain embodiments, R₁-R₄ are hydrogen, X is —(CH₂)ₙ—, wherein, n is 1, 2, or 3, and R₅ is haloalkyl (e.g. CF₃).

In certain embodiments, R₁-R₄ are hydrogen, X is —(CH₂)ₙ—, wherein, n is 1, 2, or 3, and R₅ is methoxy (—OCH₃).

In certain embodiments, R₁-R₃ are each independently selected from the group consisting of hydrogen, halogen (e.g. F, Cl, Br, and I), haloalkyl (e.g. CF₃), R₄ is hydrogen, X is —(CH₂)ₙ—, wherein, n is 1, 2, or 3, and R₅ is cyano (—CN).

In certain embodiments, R₁-R₃ are each independently selected from the group consisting of hydrogen, halogen (e.g. F, Cl, Br, and I), haloalkyl (e.g. CF₃), R₄ is methyl (—CH₃), X is —(CH₂)ₙ—, wherein, n is 1, 2, or 3, and R₅ is cyano (—CN).

In certain embodiments, R₁-R₃ are hydrogen, R₄ is methyl (—CH₃), X is —(CH₂)ₙ—, wherein, n is 1, 2, or 3, and R₅ is cyano (—CN).

In certain embodiments, R₁-R₃ are each independently selected from the group consisting of hydrogen, halogen (e.g. F, Cl, Br, and I), haloalkyl (e.g. CF₃), R₄ is hydrogen, X is —(CH₂)ₙ—, wherein, n is 1, 2, or 3, and R₅ is a halogen (e.g. F, Cl, Br, and I).

In certain embodiments, R₁-R₃ are each independently selected from the group consisting of hydrogen, halogen (e.g. F, Cl, Br, and I), haloalkyl (e.g. CF₃), R₄ is methyl (—CH₃), X is —(CH₂)ₙ—, wherein, n is 1, 2, or 3, and R₅ is a halogen (e.g. F, Cl, Br, and I).

In certain embodiments, R₁-R₃ are hydrogen, R₄ is methyl (—CH₃), X is —(CH₂)ₙ—, wherein, n is 1, 2, or 3, and R₅ is a halogen (e.g. F, Cl, Br, and I).

In certain embodiments, R₁-R₃ are each independently selected from the group consisting of hydrogen, halogen (e.g. F, Cl, Br, and I), haloalkyl (e.g. CF₃), R₄ is hydrogen, X is —(CH₂)ₙ—, wherein, n is 1, 2, or 3, and R₅ is a haloalkyl (e.g. CF₃).

In certain embodiments, R₁-R₃ are each independently selected from the group consisting of hydrogen, halogen (e.g. F, Cl, Br, and I), haloalkyl (e.g. CF₃), R₄ is methyl (—CH₃), X is —(CH₂)ₙ—, wherein, n is 1, 2, or 3, and R₅ is a haloalkyl (e.g. CF₃).

In certain embodiments, R₁-R₃ are hydrogen, R₄ is methyl (—CH₃), X is —(CH₂)ₙ—, wherein, n is 1, 2, or 3, and R₅ is a haloalkyl (e.g. CF₃).

In certain embodiments, R₁-R₃ are each independently selected from the group consisting of hydrogen, halogen (e.g. F, Cl, Br, and I), haloalkyl (e.g. CF₃), R₄ is hydrogen, X is —(CH₂)ₙ—, wherein, n is 1, 2, or 3, and R₅ is a methyl (—CH₃).

In certain embodiments, R₁-R₃ are each independently selected from the group consisting of hydrogen, halogen (e.g. F, Cl, Br, and I), haloalkyl (e.g. CF₃), R₄ is methyl (—CH₃), X is —(CH₂)ₙ—, wherein, n is 1, 2, or 3, and R₅ is a methyl (—CH₃).

In certain embodiments, R₁-R₃ are hydrogen, R₄ is methyl (—CH₃), X is —(CH₂)ₙ—, wherein, n is 1, 2, or 3, and R₅ is a methyl (—CH₃).

In certain embodiments, R₁-R₃ are each independently selected from the group consisting of hydrogen, halogen (e.g. F, Cl, Br, and I), haloalkyl (e.g. CF₃), R₄ is hydrogen, X is —(CH₂)ₙ—, wherein, n is 1, 2, or 3, and R₅ is an ethynyl (—CCH).

In certain embodiments, R₁-R₃ are each independently selected from the group consisting of hydrogen, halogen (e.g. F, Cl, Br, and I), haloalkyl (e.g. CF₃), R₄ is methyl (—CH₃), X is —(CH₂)ₙ—, wherein, n is 1, 2, or 3, and R₅ is an ethynyl (—CCH).

In certain embodiments, R₁-R₃ are hydrogen, R₄ is methyl (—CH₃), X is —(CH₂)ₙ—, wherein, n is 1, 2, or 3, and R₅ is an ethynyl (—CCH).

In certain embodiments, R₁-R₃ are each independently selected from the group consisting of hydrogen, halogen (e.g. F, Cl, Br, and I), haloalkyl (e.g. CF₃), R₄ is hydrogen, X is —(CH₂)ₙ—, wherein, n is 1, 2, or 3, and R₅ is a methoxy (—OCH₃).

In certain embodiments, R₁-R₃ are each independently selected from the group consisting of hydrogen, halogen (e.g. F, Cl, Br, and I), haloalkyl (e.g. CF₃), R₄ is methyl (—CH₃), X is —(CH₂)ₙ—, wherein, n is 1, 2, or 3, and R₅ is a methoxy (—OCH₃).

In certain embodiments, R₁-R₃ are hydrogen, R₄ is methyl (—CH₃), X is —(CH₂)ₙ—, wherein, n is 1, 2, or 3, and R₅ is a methoxy (—OCH₃).

Compounds Nos. D1-D45

In certain embodiments, the compounds provided herein are selected from the group consisting of Compound Nos. D1-D21 listed in Table 1 below, including pharmaceutically acceptable solvates, pharmaceutically acceptable prodrugs, pharmaceutically acceptable salts and pharmaceutically acceptable stereoisomers thereof.

TABLE 1
Structures of Compounds Nos. D1-D21

| NO. | Structure |
|---|---|
| D1 | 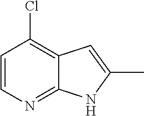 |
| D2 | 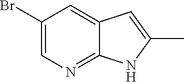 |
| D3 | 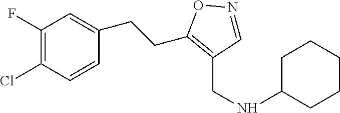 [SMILES: Fc1cc(CCc2oncc2CNC2CCCCC2)ccc1Cl] |
| D4 | 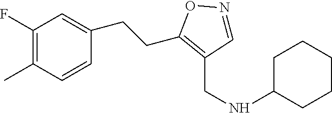 [SMILES: Cc1ccc(CCc2oncc2CNC2CCCCC2)cc1F] |
| D5 | 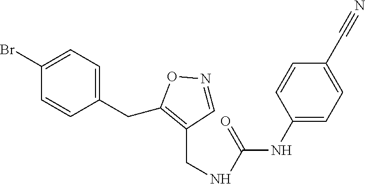 [SMILES: N#Cc1ccc(NC(=O)NCc2cnoc2Cc2ccc(Br)cc2)cc1] |
| D6 | 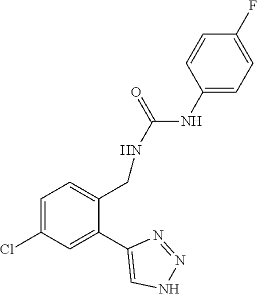 [SMILES: O=C(NCc1ccc(Cl)cc1-c1c[nH]nn1)Nc1ccc(F)cc1] |
| D7 | 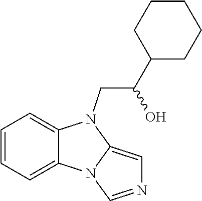 [SMILES: OC(Cn1c2ccccc2n2cncc12)C1CCCCC1] |
| D8 | 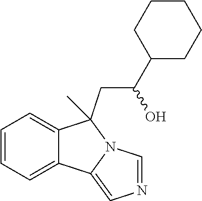 [SMILES: CC1(CC(O)C2CCCCC2)c2ccccc2-c2cncn21] |
| D9 | 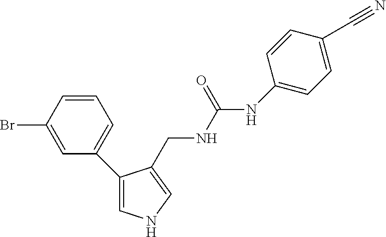 [SMILES: N#Cc1ccc(NC(=O)NCc2c[nH]cc2-c2cccc(Br)c2)cc1] |
| D10 | 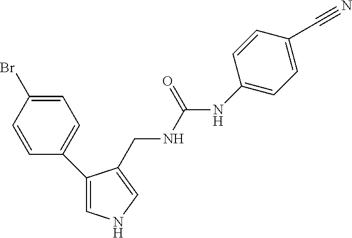 [SMILES: N#Cc1ccc(NC(=O)NCc2c[nH]cc2-c2ccc(Br)cc2)cc1] |
| D11 | 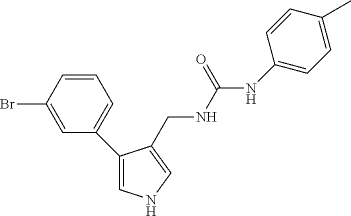 [SMILES: Cc1ccc(NC(=O)NCc2c[nH]cc2-c2cccc(Br)c2)cc1] |
| D12 | 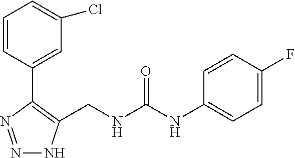 [SMILES: O=C(NCc1[nH]nnc1-c1cccc(Cl)c1)Nc1ccc(F)cc1] |
| D13 | 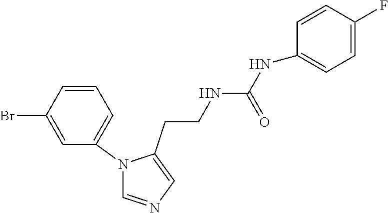 [SMILES: O=C(NCCc1cncn1-c1cccc(Br)c1)Nc1ccc(F)cc1] |
| D14 | 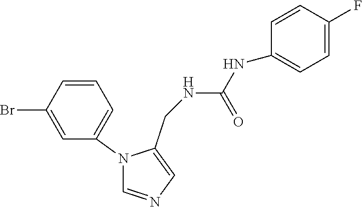 [SMILES: O=C(NCc1cncn1-c1cccc(Br)c1)Nc1ccc(F)cc1] |
| D15 | 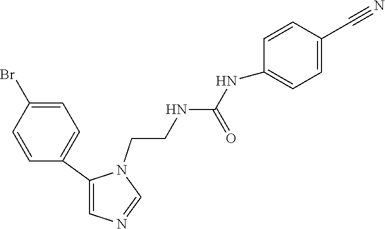 [SMILES: N#Cc1ccc(NC(=O)NCCn2cncc2-c2ccc(Br)cc2)cc1] |
| D16 | 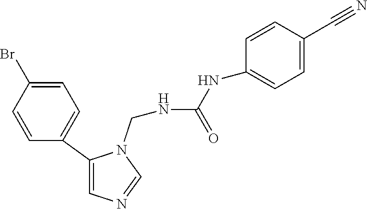 [SMILES: N#Cc1ccc(NC(=O)NCn2cncc2-c2ccc(Br)cc2)cc1] |
| D17 | 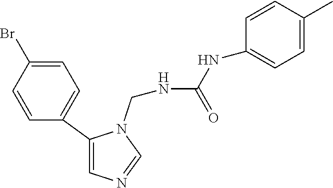 [SMILES: Cc1ccc(NC(=O)NCn2cncc2-c2ccc(Br)cc2)cc1] |
| D18 | 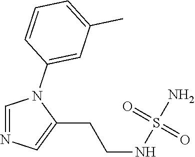 [SMILES: Cc1cccc(-n2cncc2CCNS(N)(=O)=O)c1] |
| D19 | 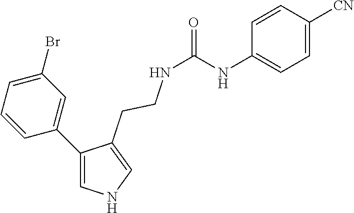 [SMILES: N#Cc1ccc(NC(=O)NCCc2c[nH]cc2-c2cccc(Br)c2)cc1] |
| D20 | 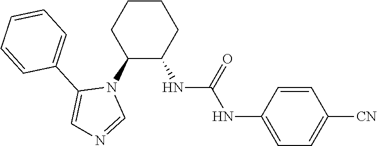 [SMILES: N#Cc1ccc(NC(=O)N[C@H]2CCCC[C@@H]2n2cncc2-c2ccccc2)cc1] |
| D21 | 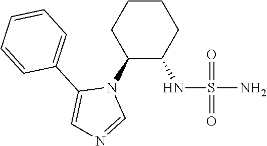 [SMILES: NS(=O)(=O)N[C@H]1CCCC[C@@H]1n1cncc1-c1ccccc1] |

In certain embodiments, the compounds provided herein are selected from the group consisting of Compounds Nos. D22-D45 listed in Table 2 below, including pharmaceutically acceptable solvates, pharmaceutically acceptable prodrugs, pharmaceutically acceptable salts and pharmaceutically acceptable stereoisomers thereof.

TABLE 2
Structures of Compounds Nos. D22-D45

| NO. | Structure |
|---|---|
| D22 | 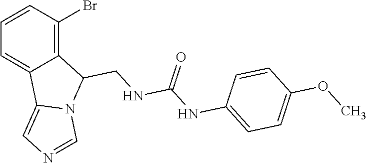 [SMILES: COc1ccc(NC(=O)NCC2c3c(Br)cccc3-c3cncn32)cc1] |
| D23 | 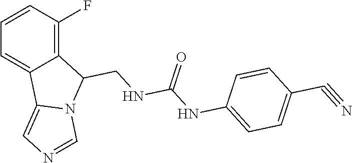 [SMILES: N#Cc1ccc(NC(=O)NCC2c3c(F)cccc3-c3cncn32)cc1] |
| D24 | 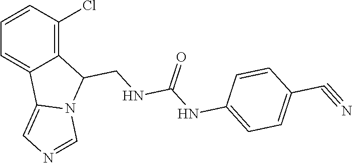 [SMILES: N#Cc1ccc(NC(=O)NCC2c3c(Cl)cccc3-c3cncn32)cc1] |
| D25 | 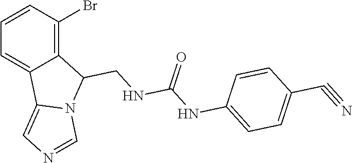 [SMILES: N#Cc1ccc(NC(=O)NCC2c3c(Br)cccc3-c3cncn32)cc1] |
| D26 | 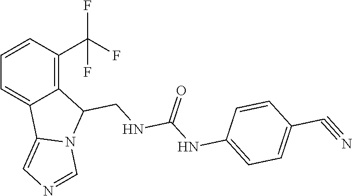 [SMILES: N#Cc1ccc(NC(=O)NCC2c3c(cccc3C(F)(F)F)-c3cncn32)cc1] |
| D27 | 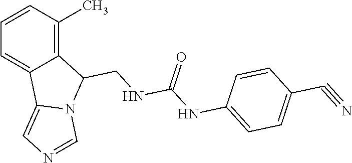 [SMILES: Cc1cccc2c1C(CNC(=O)Nc1ccc(C#N)cc1)n1cncc1-2] |
| D28 | 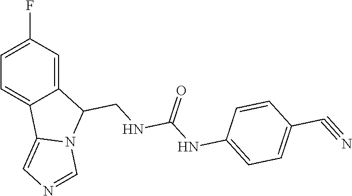 [SMILES: N#Cc1ccc(NC(=O)NCC2c3cc(F)ccc3-c3cncn32)cc1] |
| D29 | 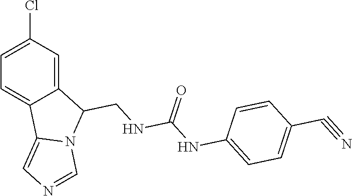 [SMILES: N#Cc1ccc(NC(=O)NCC2c3cc(Cl)ccc3-c3cncn32)cc1] |
| D30 | 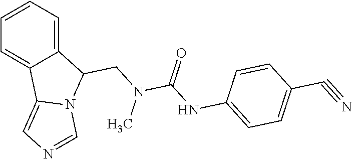 [SMILES: CN(CC1c2ccccc2-c2cncn21)C(=O)Nc1ccc(C#N)cc1] |
| D31 | 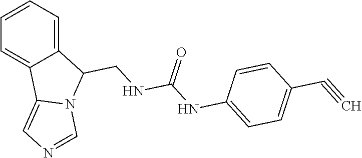 [SMILES: C#Cc1ccc(NC(=O)NCC2c3ccccc3-c3cncn32)cc1] |
| D32 | 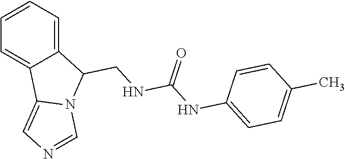 [SMILES: Cc1ccc(NC(=O)NCC2c3ccccc3-c3cncn32)cc1] |
| D33 | 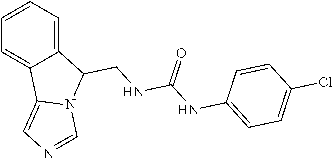 [SMILES: O=C(NCC1c2ccccc2-c2cncn21)Nc1ccc(Cl)cc1] |
| D34 | 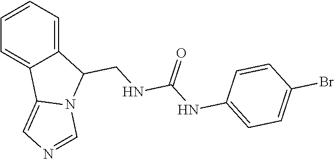 [SMILES: O=C(NCC1c2ccccc2-c2cncn21)Nc1ccc(Br)cc1] |
| D35 | 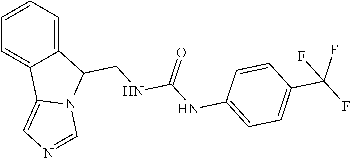 [SMILES: O=C(NCC1c2ccccc2-c2cncn21)Nc1ccc(C(F)(F)F)cc1] |
| D36 | 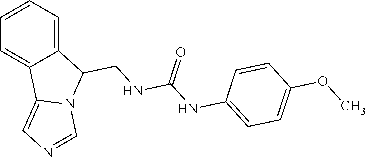 [SMILES: COc1ccc(NC(=O)NCC2c3ccccc3-c3cncn32)cc1] |
| D37 | 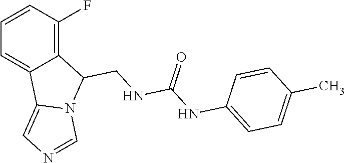 [SMILES: Cc1ccc(NC(=O)NCC2c3c(F)cccc3-c3cncn32)cc1] |
| D38 | 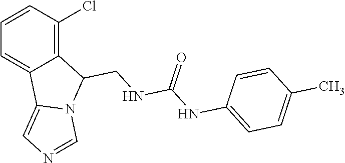 [SMILES: Cc1ccc(NC(=O)NCC2c3c(Cl)cccc3-c3cncn32)cc1] |
| D39 | 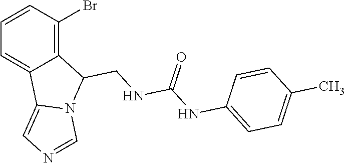 [SMILES: Cc1ccc(NC(=O)NCC2c3c(Br)cccc3-c3cncn32)cc1] |
| D40 | 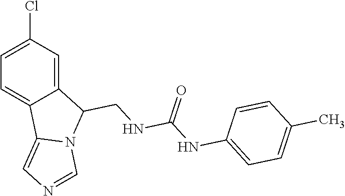 [SMILES: Cc1ccc(NC(=O)NCC2c3cc(Cl)ccc3-c3cncn32)cc1] |
| D41 | 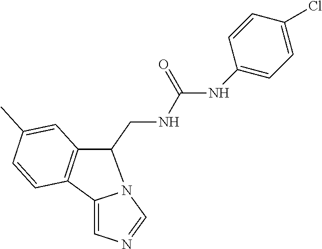 [SMILES: Cc1ccc2c(c1)C(CNC(=O)Nc1ccc(Cl)cc1)n1cncc1-2] |
| D42 | 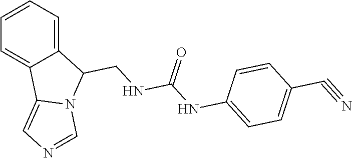 [SMILES: N#Cc1ccc(NC(=O)NCC2c3ccccc3-c3cncn32)cc1] |
| D43 | 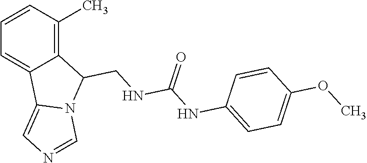 [SMILES: COc1ccc(NC(=O)NCC2c3c(C)cccc3-c3cncn32)cc1] |
| D44 | 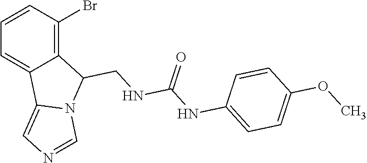 [SMILES: COc1ccc(NC(=O)NCC2c3c(Br)cccc3-c3cncn32)cc1] |
| D45 | 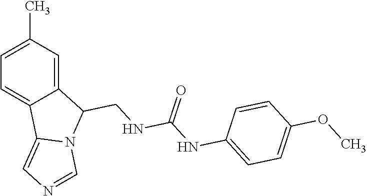 [SMILES: COc1ccc(NC(=O)NCC2c3cc(C)ccc3-c3cncn32)cc1] |
| D46 | 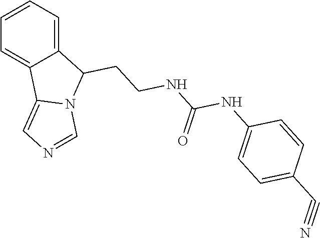 [SMILES: N#Cc1ccc(NC(=O)NCCC2c3ccccc3-c3cncn32)cc1] |

As used herein, the term “halogen” or “halo” refers to fluorine (F), chlorine (Cl), bromine (Br) or iodine (I).

As used herein, the term “haloalkyl” refers to an alkyl group wherein one or more hydrogen and/or carbon atoms are substituted with halogen atom.

As used herein, the term “substituted” refers to substitution(s) on one or more atoms, wherein each atom may be substituted with one or more substituents described above. Further examples of substitutions include, without limitation, halogen, methyl, methoxy, haloalkyl, cyano, and ethynyl.

Unless otherwise specified, all substituents intend to include optionally substituted substituents, i.e. further substituted or not. For example, an alkyl group may be an unsubstituted alkyl group, or a substituted alkyl group as defined supra.

As used herein, a compound or a composition that is “pharmaceutically acceptable” is suitable for use in contact with the tissue or organ of a biological subject without excessive toxicity, irritation, allergic response, immunogenicity, or other problems or complications, commensurate with a reasonable benefit/risk ratio. If said compound or composition is to be used with other ingredients, said compound or composition is also compatible with said other ingredients.

As used herein, the term “solvate” refers to a complex of variable stoichiometry formed by a solute (e.g., compounds provided herein) and a solvent. Such solvents may not interfere with the biological activity of the solute. Examples of suitable solvents include, but are not limited to, water, aqueous solution (e.g. buffer), methanol, ethanol and acetic acid. Preferably, the solvent used is a pharmaceutically acceptable solvent. Examples of suitable pharmaceutically acceptable solvents include, without limitation, water, aqueous solution (e.g. buffer), ethanol and acetic acid. Most preferably, the solvent used is water or aqueous solution (e.g. buffer). Examples for suitable solvates are the mono- or dihydrates or alcoholates of the compound according to the disclosure.

As used herein, pharmaceutically acceptable salts of a compound refers to any pharmaceutically acceptable acid and/or base additive salt of the compound (e.g., compounds provided herein). Suitable acids include organic and inorganic acids. Suitable bases include organic and inorganic bases. Examples of suitable inorganic acids include, but are not limited to: hydrochloric acid, hydrofluoric acid, hydrobromic acid, hydroiodic acid, sulfuric acid and boric acid. Examples of suitable organic acids include but are not limited to: acetic acid, trifluoroacetic acid, formic acid, oxalic acid, malonic acid, succinic acid, tartaric acid, maleic acid, fumaric acid, methanesulfonic acid, trifluoromethanesulfonic acid, benzoic acid, glycolic acid, lactic acid, citric acid and mandelic acid. Examples of suitable inorganic bases include, but are not limited to: ammonia, hydroxyethylamine and hydrazine. Examples of suitable organic bases include, but are not limited to, methylamine, ethylamine, trimethylamine, triethylamine, ethylenediamine, hydroxyethylamine, morpholine, piperazine and guanidine. The disclosure further provides for the hydrates and polymorphs of all of the compounds described herein.

According to some embodiments provided herein, a kit is provided that comprises one or more compounds disclosed herein or compositions or formulations thereof. In one embodiment, the kit may be used a research tool to investigate the effect of inhibition of IDO1 (indoleamine 2,3-dioxygenase-1) and of the cellular processes of the kynurenine pathway.

II. Compositions

Provided herein in certain embodiments are compositions comprising one or more of the compounds provided herein. The compounds provided herein may contain one or more chiral atoms, or may otherwise be capable of existing as two or more stereoisomers, which are usually enantiomers and/or diastereomers. Accordingly, the compositions provided herein include mixtures of stereoisomers or mixtures of enantiomers, as well as purified stereoisomers, purified enantiomers, stereoisomerically enriched mixtures, or enantiomerically enriched mixtures. The compositions provided herein also include the individual isomers of the compound represented by the structures described above as well as any wholly or partially equilibrated mixtures thereof. The compositions provided herein also include the individual isomers of the compounds represented by the structures described above as mixtures with isomers thereof in which one or more chiral centers are inverted. Also, it is understood that all tautomers and mixtures of tautomers of the structures described above are included within the scope of the structures and preferably the structures corresponding thereto.

Racemates can be resolved into the isomers mechanically or chemically by methods known per se. Diastereomers are preferably formed from the racemic mixture by reaction with an optically active resolving agent. Examples of suitable resolving agents are optically active acids, such as the D and L forms of tartaric acid, diacetyltartaric acid, dibenzoyltartaric acid, mandelic acid, malic acid, lactic acid or the various optically active camphorsulfonic acids, such as camphorsulfonic acid. Also advantageous is enantiomer resolution with the aid of a column filled with an optically active resolving agent. The diastereomer resolution can also be carried out by standard purification processes, such as, for example, chromatography or fractional crystallization.

It is also possible to obtain optically active compounds comprising the structure of the compounds disclosed herein by the methods described above by using starting materials which are already optically active.

III. Pharmaceutical Formulations

As used herein, a pharmaceutical formulation comprises a therapeutically effective amount of one or more of the compounds or compositions provided herein. In certain embodiments, the pharmaceutical formulation further comprises a pharmaceutically acceptable carrier.

As used herein, a “therapeutically effective amount,” “therapeutically effective concentration” or “therapeutically effective dose” is an amount which, as compared to a corresponding subject who has not received such amount, results in improved treatment, healing, prevention, or amelioration of a disease, disorder, or side effect, or a decrease in the rate of advancement of a disease or disorder.

This amount will vary depending upon a variety of factors, including but not limited to the characteristics of the compounds, compositions, or pharmaceutical formulations thereof (including activity, pharmacokinetics, pharmacodynamics, and bioavailability thereof), the physiological condition of the subject treated (including age, sex, disease type and stage, general physical condition, responsiveness to a given dosage, and type of medication) or cells, the nature of the pharmaceutically acceptable carrier or carriers in the formulation, and the route of administration. One skilled in the clinical and pharmacological arts will be able to determine an effective amount or therapeutically effective amount through routine experimentation, namely by monitoring a cell's or subject's response to administration of the one or more compounds disclosed herein or the pharmaceutical formulation thereof and adjusting the dosage accordingly. For additional guidance, see Remington: The Science and Practice of Pharmacy, 21st Edition, Univ. of Sciences in Philadelphia (USIP), Lippincott Williams & Wilkins, Philadelphia, Pa., 2005, which is hereby incorporated by reference as if fully set forth herein for additional guidance for determining a therapeutically effective amount.

As used herein, the term “about” refers to ±10%, ±5%, or ±1%, of the value following “about.”

A “pharmaceutically acceptable carrier” is a pharmaceutically-acceptable material, composition or vehicle, such as a liquid or solid filler, diluent, excipient, solvent or encapsulating material, involved in carrying or transporting an active ingredient from one location, body fluid, tissue, organ (interior or exterior), or portion of the body, to another location, body fluid, tissue, organ, or portion of the body. Each carrier is “pharmaceutically acceptable” in the sense of being compatible with the other ingredients, e.g., the compounds or compositions described herein or other ingredients, of the formulation and suitable for use in contact with the tissue or organ of a biological subject without excessive toxicity, irritation, allergic response, immunogenicity, or other problems or complications, commensurate with a reasonable benefit/risk ratio.

Pharmaceutically acceptable carriers are well known in the art and include, without limitation, (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) alcohol, such as ethyl alcohol and propane alcohol; (20) phosphate buffer solutions; and (21) other non-toxic compatible substances employed in pharmaceutical formulations.

The pharmaceutical formulations disclosed herein may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like.

The concentration of the one or more compounds or compositions thereof in the pharmaceutical formulations provided herein can vary widely, and will be selected primarily based on fluid volumes, viscosities, body weight and the like in accordance with the particular mode of administration selected and the biological subject's needs. For example, the concentration of the one or more compounds disclosed herein can be about 0.0001% to about 100%, about 0.001% to about 50%, about 0.01% to about 30%, about 0.1% to about 20%, about 1% to about 10% wt.

A suitable pharmaceutically acceptable carrier may be selected taking into account the chosen mode of administration, and the physical and chemical properties of the compounds.

One skilled in the art will recognize that a pharmaceutical formulation containing the one or more compounds provided herein or compositions thereof can be administered to a subject by various routes including, without limitation, orally or parenterally, such as intravenously. The composition may also be administered through subcutaneous injection, subcutaneous embedding, intragastric, topical, and/or vaginal administration. The composition may also be administered by injection or intubation.

In one embodiment, the pharmaceutical carrier may be a liquid and the pharmaceutical formulation would be in the form of a solution. In another embodiment, the pharmaceutically acceptable carrier is a solid and the pharmaceutical formulation is in the form of a powder, tablet, pill, or capsules. In another embodiment, the pharmaceutical carrier is a gel and the pharmaceutical formulation is in the form of a suppository or cream.

A solid carrier can include one or more substances which may also act as flavoring agents, lubricants, solubilizers, suspending agents, fillers, glidants, compression aids, binders or table-disintegrating agents, it can also be an encapsulating material. In powders, the carrier is a finely divided solid that is in admixture with the finely divided active ingredient. In tablets, the active-ingredient is mixed with a carrier having the necessary compression properties in suitable proportions and compacted in the shape and size desired. The powders and tablets preferably contain up to about 99% of the one or more compounds disclosed herein. Suitable solid carriers include, for example, calcium phosphate, magnesium stearate, talc, sugars, lactose, dextrin, starch, gelatin, cellulose, polyvinylpyrrolidine, low melting waxes and ion exchange resins.

Besides containing an effective amount of the one or more compounds provided herein or compositions thereof, the pharmaceutical formulations may also include suitable diluents, preservatives, solubilizers, emulsifiers, adjuvant and/or carriers.

The pharmaceutical formulation can be administered in the form of a sterile solution or suspension containing other solutes or suspending agents, for example, enough saline or glucose to make the solution isotonic, bile salts, acacia, gelatin, sorbitan monoleate, polysorbate 80 (oleate esters of sorbitol and its anhydrides copolymerized with ethylene oxide) and the like.

Additional pharmaceutical formulations will be evident to those skilled in the art, including formulations involving binding agent molecules in sustained- or controlled-delivery formulations. Techniques for formulating a variety of other sustained- or controlled-delivery means, such as liposome carriers, bio-erodible microparticles or porous beads and depot injections, are also known to those skilled in the art. See, for example, PCT/US93/0082948 which is incorporated herein by reference as if fully set forth herein for the techniques of controlled release of porous polymeric microparticles for the delivery of pharmaceutical formulations. Additional examples of sustained-release preparations include semipermeable polymer matrices in the form of shaped articles, e.g. films, or microcapsules. Sustained release matrices may include polyesters, hydrogels, polylactides, copolymers of L-glutamic acid and gamma ethyl-L-glutamate, poly (2-hydroxyethyl-methacrylate), ethylene vinyl acetate or poly-D (−)-3-hydroxybutyric acid. Sustained-release compositions also include liposomes, which can be prepared by any of several methods known in the art.

IV. Methods of Preparation

Another aspect of the disclosure relates to the preparation of the compounds disclosed herein.

In one embodiment, one or more compounds disclosed herein are synthesized according to Scheme 1, wherein R₀ represents one or more substituents as defined the same as above for R₁-R₃ of Structure I, and wherein R₅ represent one or more substituents as defined the same as above for R₅ of Structure I.

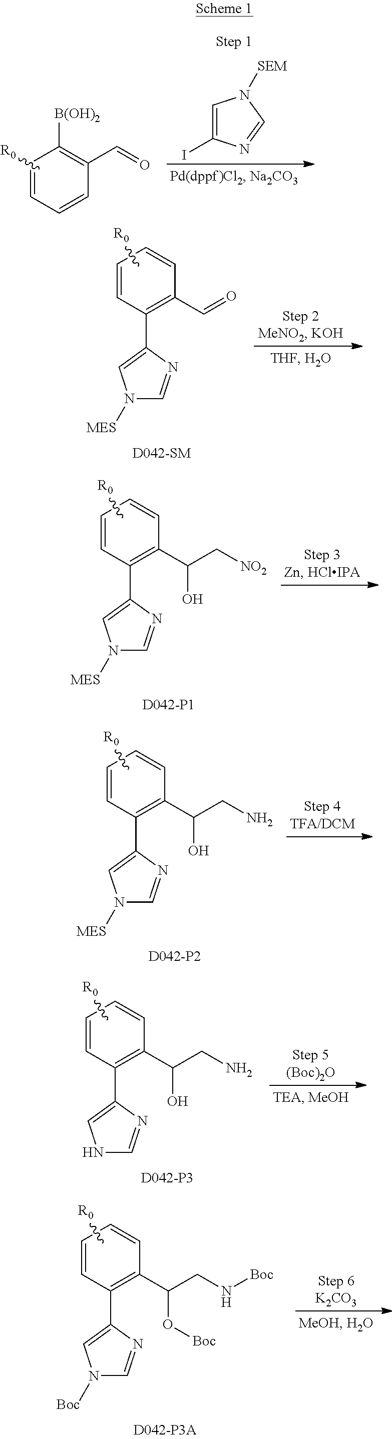

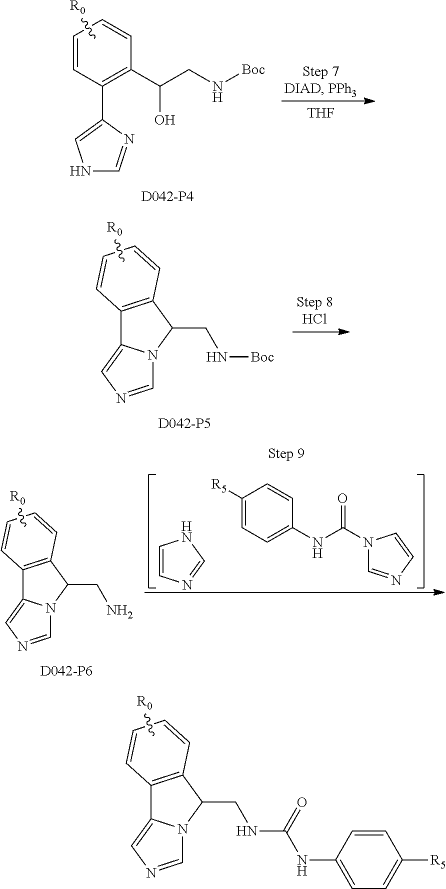

In another embodiment, one or more compounds disclosed herein can be synthesized according to a modified version Scheme 1, which yields compounds such as, 1-((5H-imidazo[5,1-a]isoindol-5-yl)methyl)-3-(4-cyanophenyl)-1-methylurea, also named herein Compound No. 30.

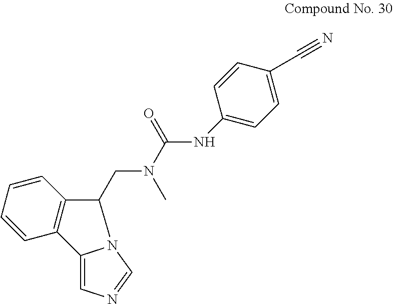 [SMILES: CN(CC1c2ccccc2-c2cncn21)C(=O)Nc1ccc(C#N)cc1]

Modification of Scheme 1 to create Compound No. 30 includes treating D-042-P6 with HCOOCH₃, followed by reduction to give the corresponding methylamine. Performing step 9 as in D-042 will provide Compound No. 30 or similar compounds as disclosed herein.

In another embodiment, one or more compounds disclosed herein are synthesized according to Scheme 2, wherein the bromine (Br) substituent of the substrate, 1,3-diromobenzene in step 3 of Scheme 2, may be replaced by a halogen (e.g. Cl, or I).

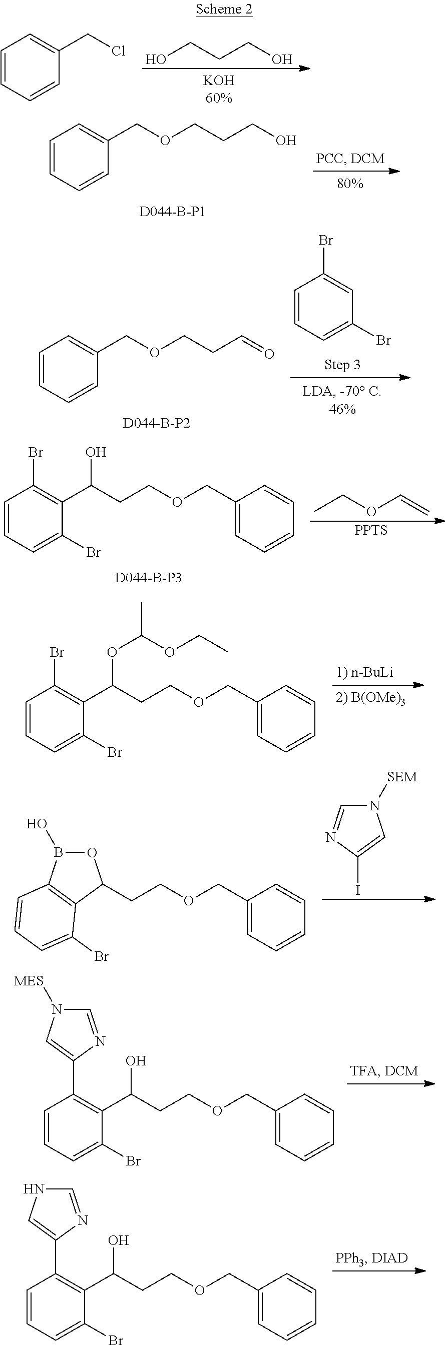

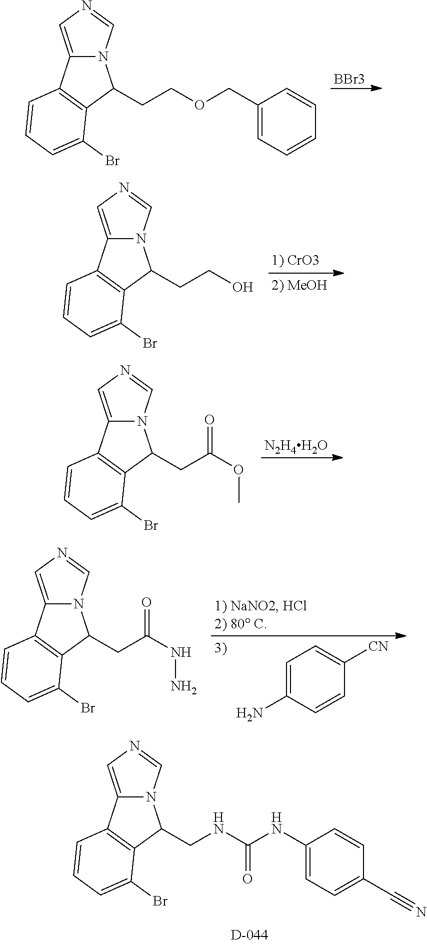

Compound No. D44 can be made according to the above Scheme 2. Benzyl chloride reacted with 1,3-propanediol gave D044-B-P1. Oxidation of D044-B-P1 with PCC yielded the aldehyde, D044-B-P2. Treatment of 1,3-diromobenzene with LDA at −70 C, followed by adding the aldehyde provided D044-B-P3. D044-B-P3 reacted with vinyl ether in the presence of PPTS gave the ketal intermediate, followed by treatment of n-BuLi first, then B(OMe)₃ provided boronic acid, suzuki coupling, deprotection with TFA, Mitsunobu reaction yielded the tricyclic bromo benzyl ether compound. Debenzylation and Jones oxidation gave bromo ester. Treatment of the ester with hydrazine, followed by oxidation, then reacted with 4-cyano-aniline provided the target compound D-044.

### EXAMPLES

### Example 1: Synthesis of Compound No. D42 [1-((5H-imidazo[5,1-a]isoindol-5-yl)methyl)-3(4-cyanophenyl)urea]

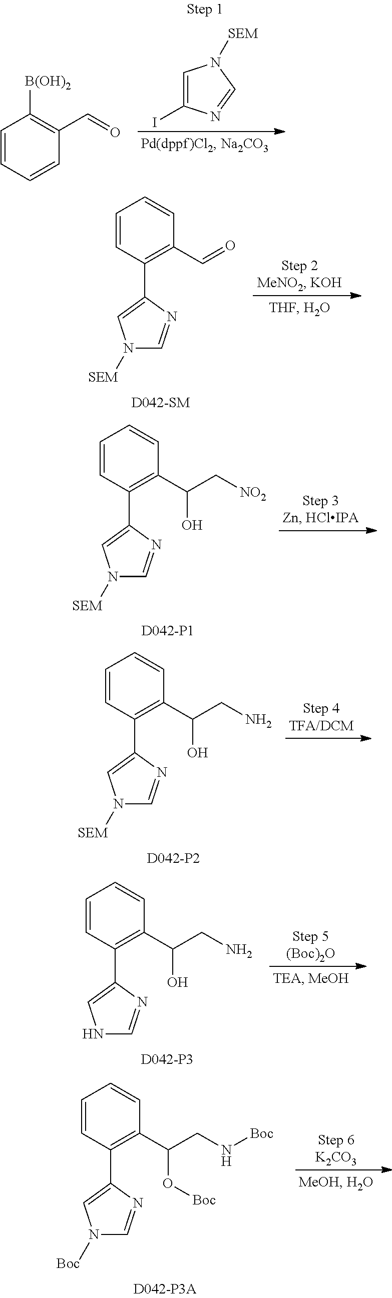

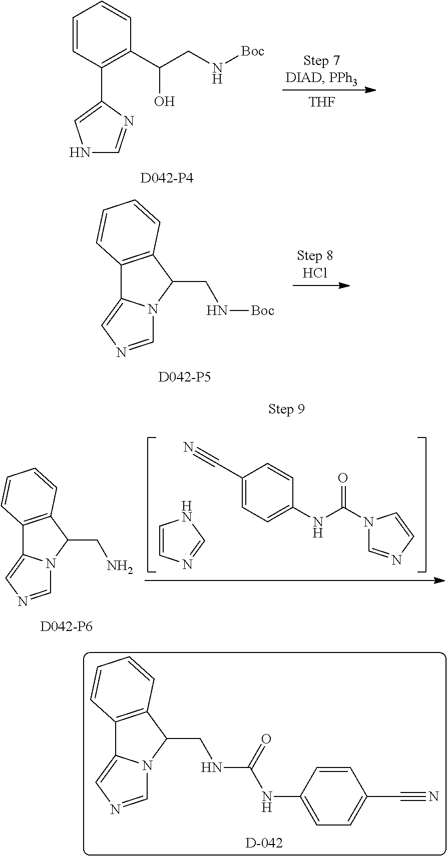

As used herein, “r.t.” “r.t” “rt.” or “rt” is room temperature.

### Step 1: The Synthesis of 2-(1-((2-(trimethylsllyl)ethoxy)methyl)-1H-imidazol-4-yl)-benzaldehyde (D42-SM)

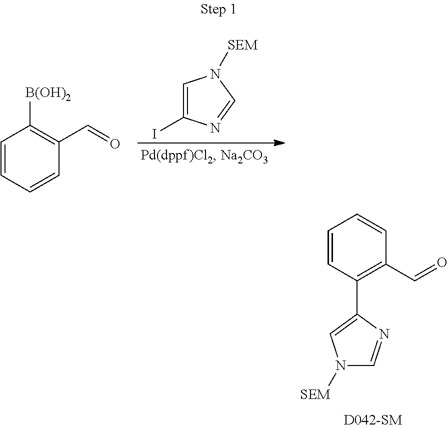

To a solution of (2-formylphenyl)boronic acid (2.001 g, 13.3 mmol, 1.2 equiv.) and 4-iodo-1-((2-(trimethylsilyl)ethoxy)methyl)-1H-imidazole (3.497 g, 10.8 mmol) in EtOH (45 mL) and toluene (45 mL) was added a solution of Na2CO3 (3.540 g, 33.4 mmol, 3.1 equiv.) in water (30 mL) and Pd(dppf)Cl2 (281 mg, 0.38 mmol, 0.036 equiv.). The flask was pump-filled with nitrogen for four times, and then heated to 76° C. After stirring at this temperature overnight, the reaction mixture was cooled to room temperature. LC-MS showed still 18-20% unconsumed starting material. To the mixture was added more Pd(dppf)Cl2 (200 mg, 0.27 mmol, 0.025 equiv.), pump-filled with nitrogen, and further stirred at 76° C. for 20 hours. The mixture was cooled to room temperature, diluted with water. The two phases were separated, and the aqueous phase was extracted with EtOAc twice. The combined organic phase was washed with water, brine, dried and concentrated. The residue was purified by column chromatography to give the desired product as brown oil (2.020 g, 6.7 mmol; yield: 62%).

### Step 2: The Synthesis of 2-nitro-1-(2-(1-((2-(trimethylsilyl)ethoxy)methyl)-1H-imidazol-4-yl)-phenyl)ethanol (D42-P1)

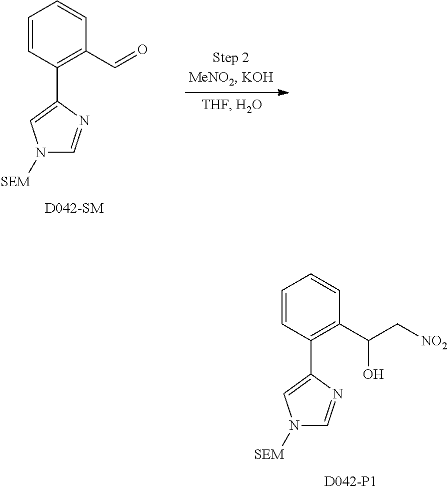 [SMILES: C[Si](C)(C)CCOCn1cnc(-c2ccccc2C=O)c1 ; C[Si](C)(C)CCOCn1cnc(-c2ccccc2C(O)C[N+](=O)[O-])c1]

To an ice-water cooled solution of D042-SM (1.129 g, 3.73 mmol) and nitromethane (1.0 mL, 18 mmol, 5.0 equiv.) in THF (25 mL) was added aqueous KOH (314 mg in 4 mL of water, 5.6 mmol, 1.5 equiv.) dropwise. The mixture was then warmed to room temperature. After stirring for 6 hours, more nitromethane (0.4 mL, 7.2 mmol, 2.0 equiv.) and KOH (118 mg, 2.1 mmol, 0.6 equiv.) was added, and the mixture was stirred overnight. The reaction was quenched with acetic acid (0.4 mL) in ice-water bath. The volatiles were removed under reduced pressure, and the residue was adjusted to pH=8-9 with Na2CO3 (aq.), and extracted with EtOAc twice. The combined organic phase was washed with water, dried and concentrated to give the crude product as brown oil (1.510 g, quant. yield).

### Step 3: The Synthesis of 2-amino-1-(2-(1-((2-(trimethylsilyl)ethoxy)methyl)-1H-imidazol-4-yl)-phenyl)ethanol (D042-P2)

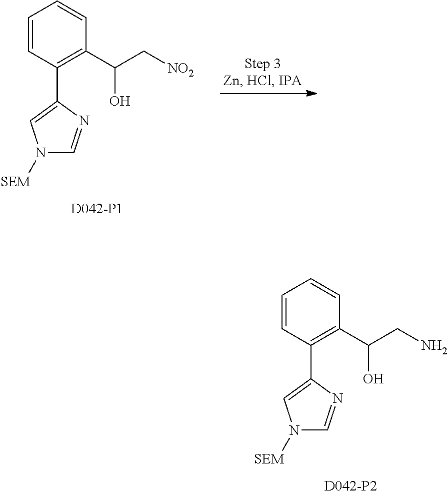 [SMILES: C[Si](C)(C)CCOCn1cnc(-c2ccccc2C(O)C[N+](=O)[O-])c1 ; C[Si](C)(C)CCOCn1cnc(-c2ccccc2C(O)CN)c1]

To an ice-water cooled solution of the crude D042-P1 (1.510 g, 3.73 mmol) in iso-propanol (IPA) (44 mL) was added water (8.5 mL) and 6M HCl (7.0 mL, 42 mmol, 11 equiv.). To the mixture was added zinc powder (1.658 g, 25.4 mmol, 6.8 equiv.) with vigorous stirring. LC-MS showed complete conversion after 20 min. Excess zinc powder was removed by filtration, and the filtrate was concentrated to remove IPA. The residue was basified with NaOH (aq.) to pH>10, treated with 5% Na2EDTA (aq.), and extracted with EtOAc for three times. The combined organic phase was washed with Na2EDTA (aq.), water and brine successively, dried and concentrated to give the crude product (1.449 g, quant. yield) as orange oil.

### Steps 4 & 5: The Synthesis of Tert-Butyl 4-(2-(2,2,11,11-tetramethyl-4,9-dioxo-3,8,10-trioxa-5-azadodecan-7-yl)phenyl)-1H-imidazole-1-carboxylate (D042-P3A)

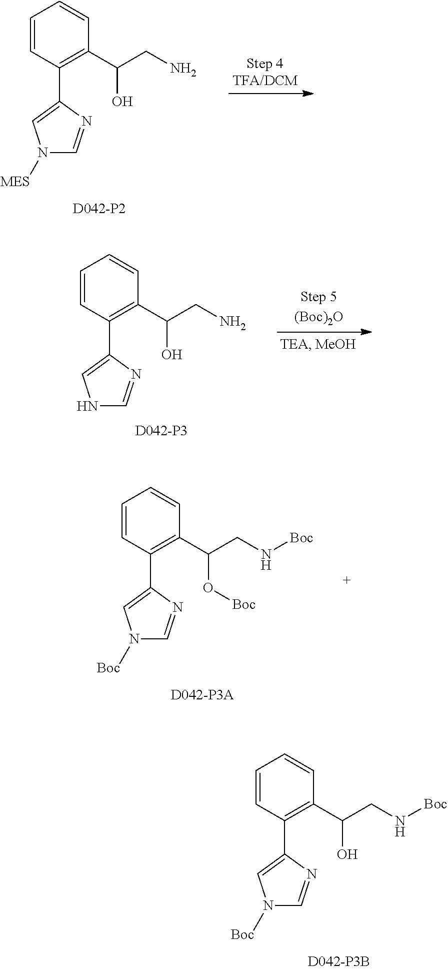

To a solution of the crude D042-P2 (1.449 g, 3.73 mmol) in dichloromethane (12 mL) was added TFA (4.0 mL), and stirred overnight at 35° C. LC-MS showed complete conversion. After removing the volatiles under reduced pressure, the residue was diluted with water, adjusted to pH>10 with NaOH (aq.), and then concentrated to dryness. The residue was re-dissolved in MeOH (40 mL) and treated with LiOH.H2O (191 mg, 4.55 mmol, 1.2 equiv.) at 35° C. overnight. The reaction mixture was adjusted to pH=8-9 with 4M HCl/MeOH, and treated with (Boc)2O (2.5 mL) at room temperature for 7-8 hours. LC-MS showed mainly the tri-Boc product D042-P3A and diBoc product D042-P3B. The reaction mixture was worked up as usual, and column chromatography purification gave both products as white or pale yellow foam, D042-P3A (292 mg, 0.58 mmol, 16% for three steps) and D042-P3B (499 mg, 1.23 mmol, 33% for three steps).

### Step 6: The Synthesis of Tert-Butyl (2-(2-(1H-imidazol-4-yl)phenyl)-2-hydroxyethyl)carbamate (D042-P4)

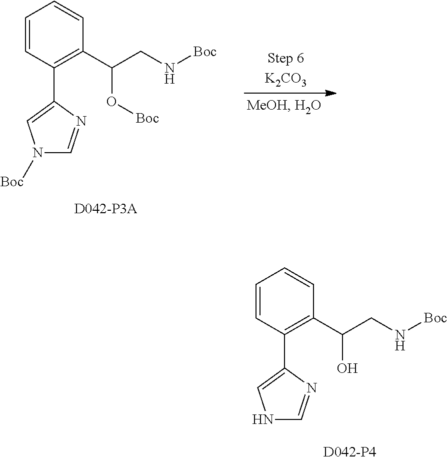 [SMILES: CC(C)(C)OC(=O)NCC(OC(=O)OC(C)(C)C)c1ccccc1-c1cn(C(=O)OC(C)(C)C)cn1 ; CC(C)(C)OC(=O)NCC(O)c1ccccc1-c1c[nH]cn1]

To a solution of D042-P3A (292 mg, 0.58 mmol) and D042-P3B (499 mg, 1.23 mmol) in MeOH (23 mL) was added K2CO3 (2.201 g, 8.7 equiv.). The mixture was stirred at 25° C. for 8 hours. After removal of MeOH, the residue was partitioned between water and EtOAc. The organic phase was washed with brine, dried and concentrated to give the crude product as a pale yellow solid (598 mg, quant. yield).

### Step 7: The Synthesis of Tert-Butyl ((5H-midazo[5,1-a]isolndol-5-yl)methyl)carbamate (D042-P5)

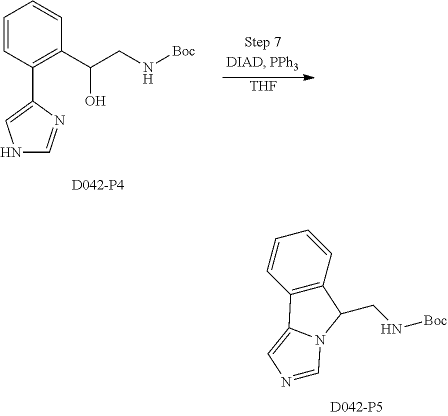 [SMILES: CC(C)(C)OC(=O)NCC(O)c1ccccc1-c1c[nH]cn1 ; CC(C)(C)OC(=O)NCC1c2ccccc2-c2cncn21 ; "THF"]

To an ice-cooled suspension of the crude D042-P4 (598 mg, 1.82 mmol) and PPh3 (801 mg, 3.05 mmol, 1.68 equiv.) in THF (35 mL) was added DIAD (0.58 mL, 2.95 mmol, 1.62 equiv.) dropwise. After stirring at 0-5 C for 30 min, the reaction was quenched with water. Organic solvent was removed, and the residue was extracted with EtOAc twice. The combined organic phase was extracted with 5% oxalic acid (aq.) twice. The combined aqueous phase was washed with EtOAc twice, and adjusted to pH>12 with NaOH (aq.). The resultant turbid mixture was extracted with dichloromethane (DCM) twice. The combined DCM phase was dried and concentrated to give the desired product as a pale yellow solid (365 mg, 1.28 mmol, 70% for two steps).

### Step 8: The Synthesis of (5H-imidazo[5,1-a]isoindol-5-yl)methanamine (D042-P6)

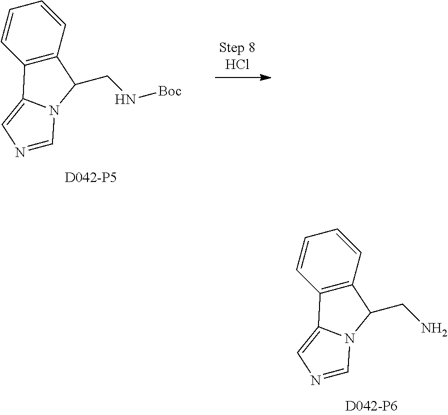 [SMILES: CC(C)(C)OC(=O)NCC1c2ccccc2-c2cncn21 ; NCC1c2ccccc2-c2cncn21]

D042-P5 (365 mg, 1.28 mmol) was dissolved in 4M HCl/MeOH (16 mL) and stirred at room temperature for 5 hours. LC-MS showed complete conversion. The solvent was removed, and the residue was co-evaporated with 1,4-dioxane twice to give the hydrochloride as a yellow solid (368 mg). To a solution of this hydrochloride (368 mg) in MeOH (20 mL) was added AmberlitelRN-78 OH-form ion-exchange resin (3.646 g). After stirring at room temperature for 30 min, the resin was removed by filtration, and the filtrate was concentrated to give the desired product as brown oil (215 mg, 1.16 mmol; yield: 91%).

### Step 9: The Synthesis of 1-((5H-imidazo[5,1-a]isoindol-5-yl)methyl)-3-(4-cyanophenyl)urea (D-042)

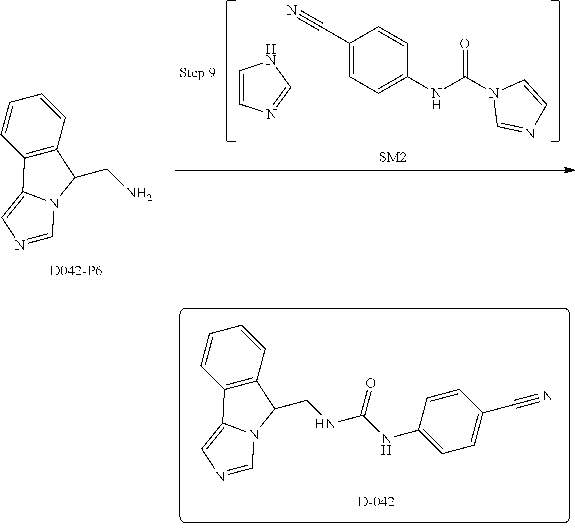

To a suspension of 4-aminobenzonitrile (1.030 g, 8.7 mmol) in toluene (10 mL) was added CDI (1.570 g, 9.7 mmol) and one drop of DBU. After stirring at room temperature for 4 hours, LC-MS showed complete conversion. The solid was collected by filtration, and dried in vacuum to give the N-(4-cyanophenyl)-1H-imidazole-1-carboxamide/imidazole mixture SM2 (2.443 g) in quantitative yield.

To a solution of D042-P6 (55 mg, 0.30 mmol) in THF (5 mL) was added TEA (0.05 mL, 0.36 mmol, 1.2 equiv.) and SM2 (94 mg, 0.34 mmol, 1.1 equiv.). After stirring at room temperature for 1 hour, the reaction was quenched with water (3 mL), and further stirred for 30 min. The volatiles were removed, and the residue was extracted with EA twice. The combined organic phase was washed with water, dried and concentrated. The residue was triturated with THF/MTBE (1:2, 5 mL) to give the desired product as a pale brown solid (48 mg, 0.15 mmol; yield: 49%). Purity: 95.5% (264 nm). LC-MS: m/z=330.4 [M+H]⁺.

¹H NMR (400 MHz, DMSO-d₆) δ 3.52-3.62 (m, 1H), 3.90-4.00 (m, 1H), 5.45 (t, J=5.3 Hz, 1H), 6.53 (t, J=5.7 Hz, 1H), 7.15 (s, 1H), 7.30 (td, J=7.5, 0.5 Hz, 1H), 7.41 (t, J=7.5 Hz, 1H), 7.52 (d, J=8.8 Hz, 2H), 7.56 (d, J=7.6 Hz, 1H), 7.62 (d, J=7.6 Hz, 1H), 7.65 (d, J=8.8 Hz, 2H), 7.89 (s, 1H), 9.10 (s, 1H).

### Example 2: Synthesis of Compound No. D46

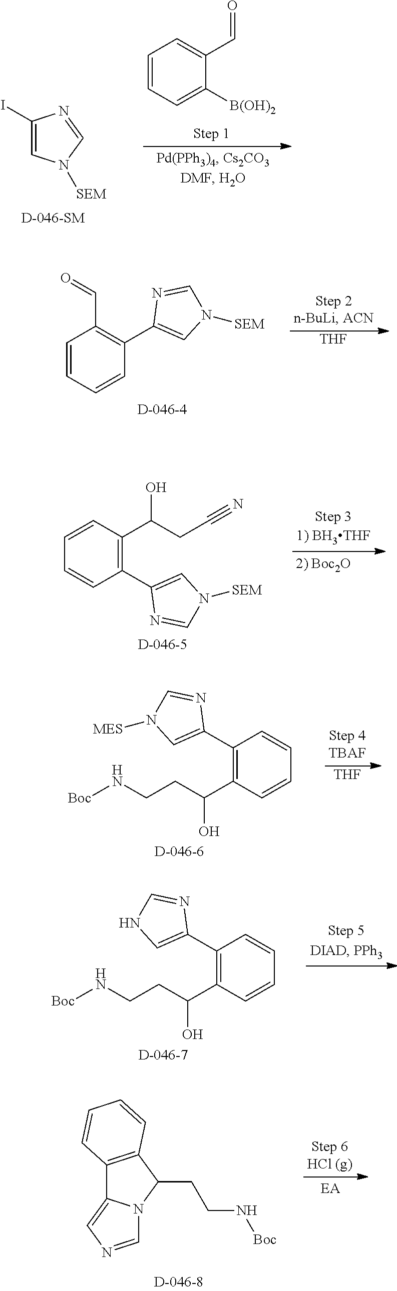

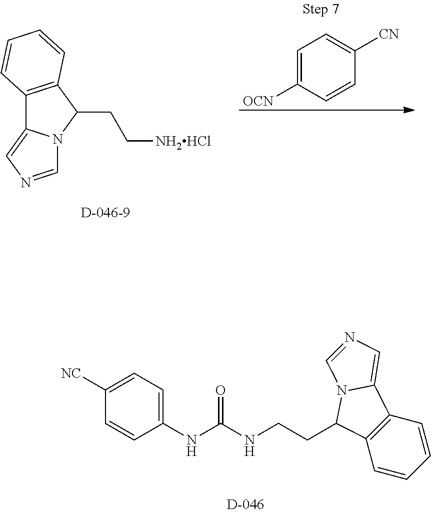

### Step 1: The Synthesis of 2-(1-((2-(trimethylsilyl)ethoxy)methyl)-1H-imidazol-4-yl)benzal-dehyde (D-046-4)

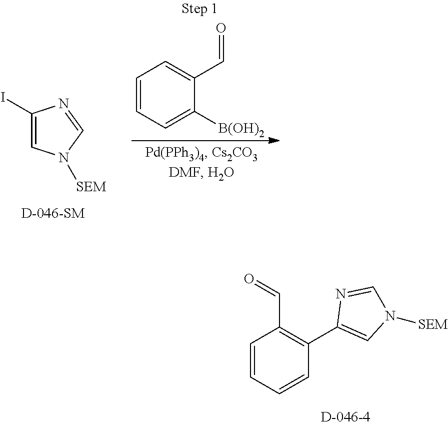 [SMILES: C[Si](C)(C)CCOCn1cnc(I)c1 ; O=Cc1ccccc1B(O)O ; C[Si](C)(C)CCOCn1cnc(-c2ccccc2C=O)c1]

To a mixture of D-046-SM (850 mg, 2.62 mmol), Cs2CO3 (1280 mg, 3.93 mmol) and (2-formylphenyl)boronic acid (471 mg, 3.14 mmol) in DMF/H2O (1:1, 10 mL) was added Pd(PPh3)4 (60 mg, 0.052 mmol) under nitrogen. The reaction mixture was stirred at 85° C. overnight. The reaction mixture was cooled to room temperature, diluted with brine and extracted with EtOAc. The combined organic layer was washed with brine, dried over Na2SO4 and concentrated. The residue was purified by silica gel column chromatography to give the desired product (400 mg, 1.32 mmol, yield: 50%).

### Step 2: The Synthesis of 3-hydroxy-3-(2-(1-((2-(trimethylsilyl)ethoxy)methyl)-1H-imidazol-4-yl)phenyl)propanenitrile (D-046-5)

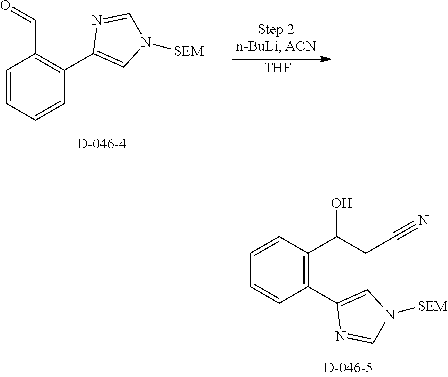 [SMILES: C[Si](C)(C)CCOCn1cnc(-c2ccccc2C=O)c1 ; C[Si](C)(C)CCOCn1cnc(-c2ccccc2C(O)CC#N)c1 ; "THF"]

To a solution of n-BuLi (2.5 M/hexane, 0.8 mL, 2.0 mmol) in THF (5 mL) was added ACN (65 mg, 1.6 mmol) at −70° C. under N₂. After stirring at −70° C. for 0.5 h, D-046-4 (400 mg, 1.32 mmol) was added. The resultant mixture was warmed to room temperature and stirred for 2 h. After completion, the reaction was quenched with brine and extracted with EtOAc. the combined organic layer was washed with brine, dried over MgSO₄ and concentrated to give the crude product of D-046-5 (500 mg).

### Step 3: The Synthesis of Tert-Butyl (3-hydroxy-3-(2-(1-((2-(trimethylsilyl)ethoxy)methyl)-1H-imidazol-4-yl)phenyl)propyl)carbamate (D-046-6)

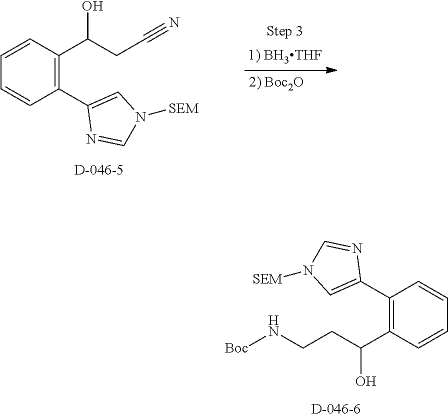 [SMILES: C[Si](C)(C)CCOCn1cnc(-c2ccccc2C(O)CC#N)c1 ; CC(C)(C)OC(=O)NCCC(O)c1ccccc1-c1cn(COCC[Si](C)(C)C)cn1]

To a solution of crude D-046-5 (500 mg, 1.32 mmol) in dry THF (10 mL) was added BH₃-THF (1M/THF, 2.0 mL, 2.0 mmol) at 0° C. The mixture was stirred at room temperature for 1 h. After completion of the reaction, Boc2O (654 mg, 3.0 mmol) and TEA (304 mg, 3.0 mmol) were added to the mixture and stirred for another 1 h. After completion of the reaction, the mixture was diluted with brine and extracted with EtOAc. The combined organic layer was concentrated and the residue was purified by silica gel column chromatography to give the product (350 mg, 0.78 mmol, yield: 59.0% for two steps).

### Step 4: The Synthesis of Tert-Butyl (3-(2-(1H-imidazol-4-yl)phenyl)-3-hydroxypropyl)-carbamate (D-046-7)

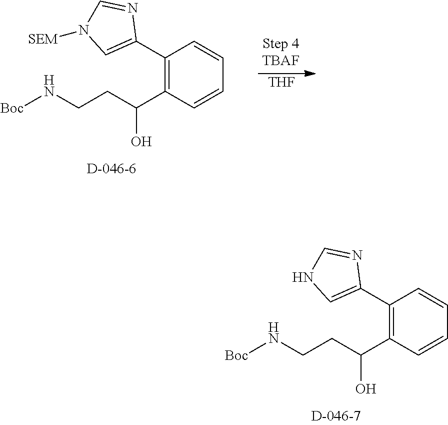 [SMILES: CC(C)(C)OC(=O)NCCC(O)c1ccccc1-c1cn(COCC[Si](C)(C)C)cn1 ; CC(C)(C)OC(=O)NCCC(O)c1ccccc1-c1c[nH]cn1 ; "THF"]

To a solution of D-046-6 (350 mg, 0.78 mmol) in THF (10 mL) was added TBAF (1M/THF, 1.6 mL, 1.60 mmol). The mixture was hated to 30° C. and stirred. After completion of the reaction, the mixture was diluted with brine and extracted with EtOAc. The combined organic phase was washed with water and concentrated to give the crude product (280 mg).

### Step 5: The Synthesis of Tert-Butyl (2-(5H-imidazo[5,1-a]isoindol-5-yl)ethyl)carbamate (D-046-8)

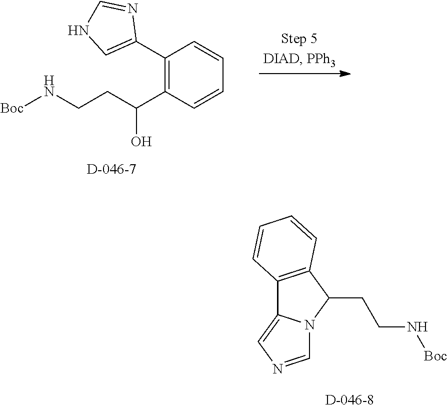 [SMILES: CC(C)(C)OC(=O)NCCC(O)c1ccccc1-c1c[nH]cn1 ; CC(C)(C)OC(=O)NCCC1c2ccccc2-c2cncn21]

To a solution of the crude D-046-7 (280 mg, 0.78 mmol) in THF (10 mL) was added triphenylphosphine (409 mg, 1.56 mmol) and DIAD (315 mg, 1.56 mmol) under N₂ and stirred at room temperature. After completion of the reaction, the mixture was diluted with brine and extracted with EtOAc. the combined organic phase was concentrated to give the crude product (1000 mg).

### Step 6: The Synthesis of 2-(5H-imidazo[5,1-a]isoindol-5-yl)ethanamine hydrochloride (D-046-9)

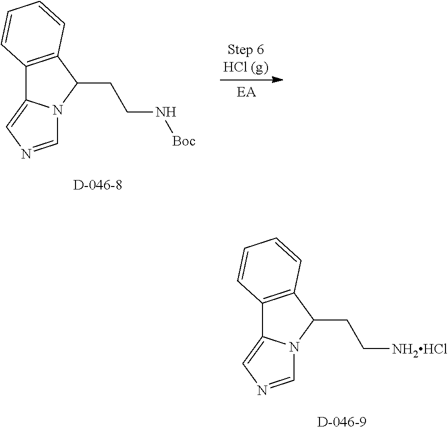

Into dry EtOAc (10 mL) cooling in an ice-water bath was bubbled HCl gas until saturation. To this solution was added the crude D-046-8 (1000 mg, 0.78 mmol). The resultant slurry was warmed to room temperature and stirred for 0.5 hour. The so-formed solid was collected by filtration and washed with EtOAc to afford the desired product (100 mg, 0.426 mmol; yield: 54.6% for three steps).

### Step 7: The Synthesis of 1-(2-(5H-imidazo[5,1-a]isoindol-5-yl)ethyl)-3-(4-cyanophenyl)urea (D-046)

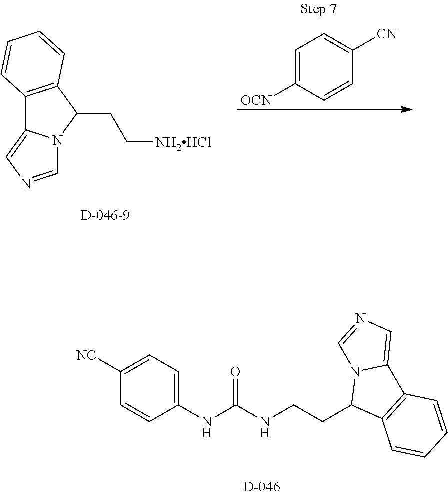

To To a solution of D-046-5 (100 mg, 0.426 mmol) in dichloromethane (10 mL) was added TEA (60 mg, 0.60 mmol) and freshly prepared 4-isocyanatobenzonitrile (56 mg, 0.39 mmol). The mixture was stirred at room temperature. After completion of the reaction, the mixture was diluted with water, and extracted with dichloromethane. The combined organic phase was concentrated. The residue was purified by prep-HPLC to afford the desired product (6 mg, 0.02 mmol, yield: 4.5%). Purity: 98% (LC-MS, 254 nm); LC-MS: m/z=344.1 ([M+H]⁺).

¹H NMR (400 MHz, DMSO-d₆) δ 1.99-2.08 (m, 2H), 3.08-3.21 (m, 2H), 5.44 (t, J=5.6 Hz, 1H), 6.54 (t, J=5.2 Hz, 1H), 7.26-7.37 (m, 2H), 7.42 (t, J=7.4 Hz, 1H), 7.53-7.60 (m, 3H), 7.62-7.70 (m, 3H), 8.26 (s, 1H), 9.14 (s, 1H).

### Example 3: IDO1 (indoleamine 2,3-dioxygenase-1) Inhibitor Screening Assay

Method

Detection of IDO1 Inhibition.

IDO1 (indoleamine 2,3-dioxygenase-1) catalyzes the oxidative cleavage of the pyrrole ring of tryptophan to yield N′-formylkynurenine. The amount of N′-formylkynurenine product is able to be detected by the absorption at the wavelength of 321 nm. IDO1 inhibitor Screening Assay kit (BPS Bioscience) was used to test the compound. The assay was performed as described in the assay protocol.

IDO1 reaction solution (90 μl) was aliquoted into each well of a 96-well plate. 5 μl of the compound solution in 10% DMSO was added to each well. 5 μl 10% DMSO was added to the positive control and blank wells. Human IDO1 His-Tag was diluted to 40 ng/μl in 1×IDO1 Assay buffer. The reaction was initiated by adding 5 μl of diluted IDO1. In the blank well, only 1×IDO1 assay buffer was added instead. After incubating at room temperature for 3 hours, the absorption was measured at the wavelength of 321 nm using a microplate reader.

The Relative Activity and IC50 Calculations.

The test compound decreased the product (N′-formylkynurenine) produced by IDO1 by X-fold. The control solution (DMSO) also decreased the product (N′-formylkynurenine) by Y-fold. The relative activity, I, was the ratio of former to later, i.e. X/Y, expressed in percentile. The IC50 was the concentration of the compounds when X/Y equals 50 and calculated based on the dose-response curve.

Results

The IC50 of the compounds is shown in the Table 3.

TABLE 3
IC50 (μM) for IDO1 Inhibition

| | | IDO1 |
|---|---|---|
| | | IC50 |
| No. | Structure | (μM) |
| D1 | 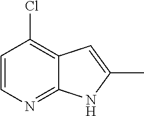 | N.D. |
| D2 | 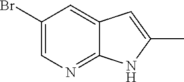 | N.D. |
| D3 | 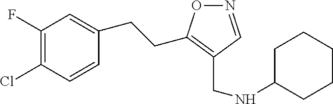 [SMILES: Fc1cc(CCc2oncc2CNC2CCCCC2)ccc1Cl] | N.D. |
| D4 | 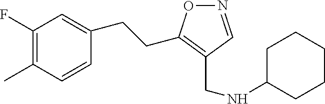 [SMILES: Cc1ccc(CCc2oncc2CNC2CCCCC2)cc1F] | N.D. |
| D5 | 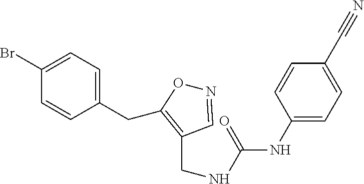 [SMILES: N#Cc1ccc(NC(=O)NCc2cnoc2Cc2ccc(Br)cc2)cc1] | N.D. |
| D6 | 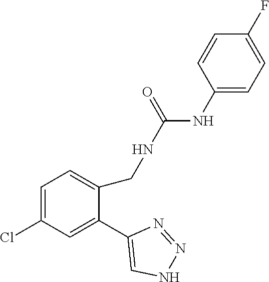 [SMILES: O=C(NCc1ccc(Cl)cc1-c1c[nH]nn1)Nc1ccc(F)cc1] | N.D. |
| D7 | 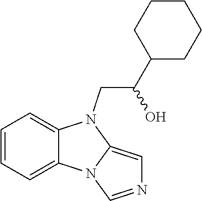 [SMILES: OC(Cn1c2ccccc2n2cncc12)C1CCCCC1] | N.D. |
| D8 | 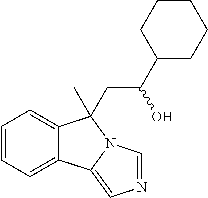 [SMILES: CC1(CC(O)C2CCCCC2)c2ccccc2-c2cncn21] | N.D. |
| D9 | 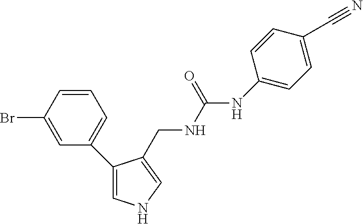 [SMILES: N#Cc1ccc(NC(=O)NCc2c[nH]cc2-c2cccc(Br)c2)cc1] | N.D. |
| D10 | 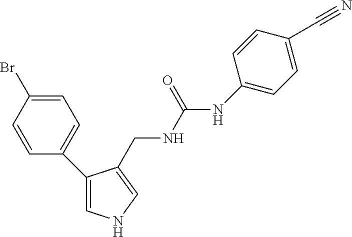 [SMILES: N#Cc1ccc(NC(=O)NCc2c[nH]cc2-c2ccc(Br)cc2)cc1] | N.D. |
| D11 | 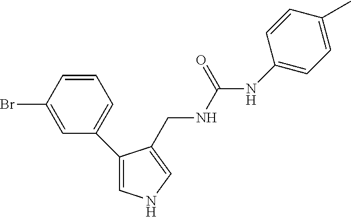 [SMILES: Cc1ccc(NC(=O)NCc2c[nH]cc2-c2cccc(Br)c2)cc1] | N.D. |
| D12 | 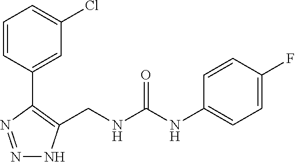 [SMILES: O=C(NCc1[nH]nnc1-c1cccc(Cl)c1)Nc1ccc(F)cc1] | N.D. |
| D13 | 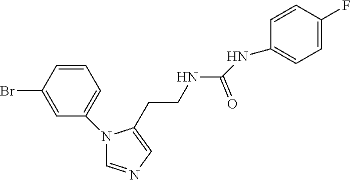 [SMILES: O=C(NCCc1cncn1-c1cccc(Br)c1)Nc1ccc(F)cc1] | N.D. |
| D14 | 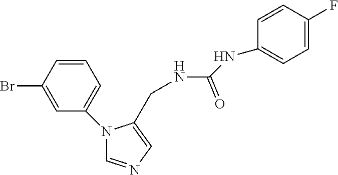 [SMILES: O=C(NCc1cncn1-c1cccc(Br)c1)Nc1ccc(F)cc1] | N.D. |
| D15 | 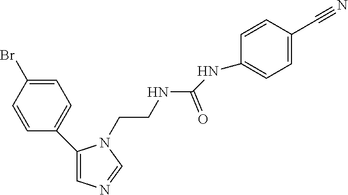 [SMILES: N#Cc1ccc(NC(=O)NCCn2cncc2-c2ccc(Br)cc2)cc1] | N.D. |
| D16 | 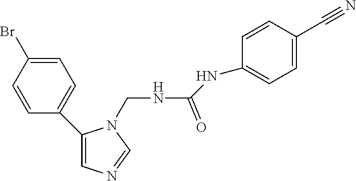 [SMILES: N#Cc1ccc(NC(=O)NCn2cncc2-c2ccc(Br)cc2)cc1] | N.D. |
| D17 | 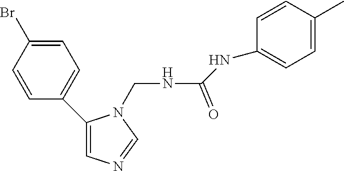 [SMILES: Cc1ccc(NC(=O)NCn2cncc2-c2ccc(Br)cc2)cc1] | N.D. |
| D42 | 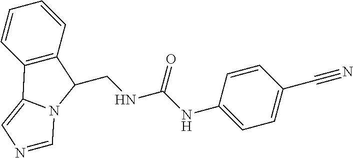 [SMILES: N#Cc1ccc(NC(=O)NCC2c3ccccc3-c3cncn32)cc1] | 0.061 |
| D46 | 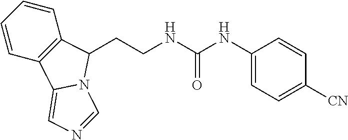 [SMILES: N#Cc1ccc(NC(=O)NCCC2c3ccccc3-c3cncn32)cc1] | 0.325 |
| D18 | 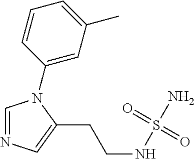 [SMILES: Cc1cccc(-n2cncc2CCNS(N)(=O)=O)c1] | N.D. |
| D19 | 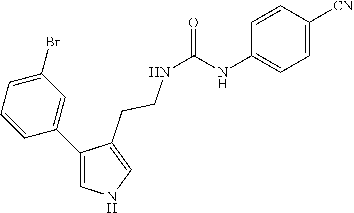 [SMILES: N#Cc1ccc(NC(=O)NCCc2c[nH]cc2-c2cccc(Br)c2)cc1] | N.D. |
| D20 | 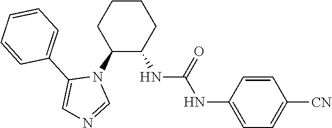 [SMILES: N#Cc1ccc(NC(=O)N[C@H]2CCCC[C@@H]2n2cncc2-c2ccccc2)cc1] | N.D. |
| D21 | 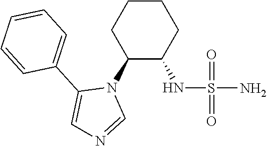 [SMILES: NS(=O)(=O)N[C@H]1CCCC[C@@H]1n1cncc1-c1ccccc1] | N.D. |

N.D. = Not Detected

### Example 4: IDO2 (indoleamine 2,3-dioxygenase-1) Inhibitor Screening Assay

Method

To evaluate the selectivity of IDO inhibition for Compounds Nos. D42 and D46, an assay to measure activity against IDO2 was performed. The IDO2 Inhibitor Screening Assay kit (BPS Bioscience) was used for IDO2 test. The protocol is similar to IDO1 assay. IDO2 reaction solution (90 μl) was aliquoted into each well of a 96-well plate. 5 μl of compound solution in 10% DMSO was added to each well. 5 μl 10% DMSO was added to the positive control and blank wells. Human IDO2 His-Tag was diluted in 1×IDO2 Assay buffer at 400 ng/μl. The reaction was initiated by adding 5 μl diluted IDO2. 1×IDO2 assay buffer to the blank well. After incubating at room temperature for 3 hours, the absorption was measured at the wavelength of 321 nm. The IC50 was calculated as described for IDO2.

The Relative Activity and IC50 Calculations.

The test compound inhibition was measured as X-fold. The control solution (DMSO) was measured as Y-fold. The relative activity, I, was the ratio of former to later, i.e. X/Y, expressed in percentile. The IC50 was the concentration of the compounds when X/Y equals 50 and calculated based on the dose-response curve.

Results

No IDO2 inhibition was detected for Compound Nos. D42 and D46.

### Example 5: TDO Inhibition Screening Assay

Method

The TDO inhibitor Screening Assay kit (BPS Bioscience) was used to test for TDO inhibition by Compound Nos. D42 and D46. The protocol is similar to IDO1 assay. TDO reaction solution (90 μl) was aliquoted into each well of a 96-well plate. 5 μl of compound solution in 10% DMSO was added to each well. 5 μl 10% DMSO was added to the positive control and blank wells. Human TDO His-Tag was diluted in 1×TDO Assay buffer at 50 ng/μl. The reaction was initiated by adding 5 μl diluted TDO. 1×TDO assay buffer was added to the blank well. After incubating at room temperature for 1.5 hours, the absorption was measured at the wavelength of 321 nm. The IC50 was calculated as described for TDO inhibition. The relative activity and IC50 was calculated as described above in Examples 3 and 4.

Results

The results showed that D42 and D46 inhibit TDO activity. The IC₅₀ (μM) for D42 and D46 was 0.064 μM for both compounds.

### REFERENCES

The references, patents and published patent applications listed below, and all references cited in the specification above are hereby incorporated by reference in their entireties, as if fully set forth herein.
1. Physician's Desk Reference, 41st Ed., Publisher Edward R. Barnhart, N.J. (1987).
2. PCT/US93/0082948.
3. Remington: The Science and Practice of Pharmacy, 21st Edition, Univ. of Sciences in Philadelphia (USIP), Lippincott Williams & Wilkins, Philadelphia, Pa., 2005.
4. Wu, L., Smythe, A. M., Stinson, et. al. Multidrug-resistant Phenotype of Disease-oriented Panels of Human Tumor Cell Lines Used for Anticancer Drug Screening. (1992) Cancer Research 52: 3029-3034.

What is claimed is:

1. A compound comprising a structure of Structure I: 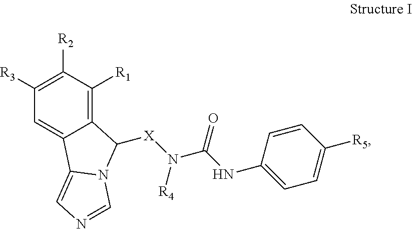
including pharmaceutically acceptable solvates, pharmaceutically acceptable prodrugs, pharmaceutically acceptable salts and pharmaceutically acceptable stereoisomers thereof, wherein:
R₁-R₃ are each independently selected from the group consisting of hydrogen, halogen (e.g. F, Cl, Br, and I), haloalkyl (e.g. CF₃), and methyl;
R₄ are each independently selected from the group consisting of hydrogen and methyl;
X is —(CH₂)ₙ—, wherein, n is 1, 2, or 3; and
R₅ is independently selected from the group consisting of halogen (e.g. F, Cl, Br, and I), haloalkyl (e.g. CF₃), methyl (—CH₃), cyano (—CN), ethynyl (—CCH), and methoxy (—OCH₃).
